(19) United States
(12) Patent Application Publication
(10) Pub. No.: US 2005/0148062 A1
(43) Pub. Date: Jul. 7, 2005

(54) Cell death related drug targets in yeast and fungi

(76) Inventors: Roland Henri Contreras, Gent (BE); Marianne Denise De Backer, Beerse (BE); Walter Herman Maria Louis Luyten, Beerse (BE); Isabelle Karin Pieter Lenaerts, Gent (BE); Bart Jozef Maria Nelissen, Beerse (BE); Rieka Josephina Reekmans, Gent (BE)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/057,823

(22) Filed: Feb. 14, 2005

Related U.S. Application Data
(62) Division of application No. 10/030,019, filed on Dec. 26, 2001.

(30) Foreign Application Priority Data
Jul. 1, 1999 (EP) 99870141.1

(51) Int. Cl. C12N 1/18; C12N 1/16; C12N 15/74; C07H 21/04

(52) U.S. Cl. 435/254.21; 435/254.22; 435/254.3; 435/483; 536/23.2

(57) ABSTRACT

The invention describes the use of the nucleic acid SEQ ID No 1 and polypeptide SEQ ID No 2 which are involved in a pathway eventually leading to programmed cell death of yeast or fungi for the preparation of a medicament for treating diseases associated with yeast or fungi or for the treatment of proliferative disorders or for preventing apoptosis in certain diseases. Methods are provided to identify compounds which selectively modulate the expression or functionality of said polypeptides in the same or a parallel pathway. Also provided are compounds as well as pharmaceutical compositions, medicaments and vaccines. The invention also comprises a new nucleic acid sequence, probes and primers derived thereof, expression vectors and host cells transformed with said vectors, polypeptides and antibodies raised against said polypeptides.

This is a divisional of application Ser. No. 10/030,019, filed Dec. 26, 2001, now ______, which claims priority from International Application Publication No. WO 01/02550 entitled “Cell Death Related Drug Targets in Yeast and Fungi”, filed Jul. 3, 2000 which claims priority from EPO Patent Application No. 99870141.1, filed Jul. 1, 1999, the contents of all of which are hereby incorporated by reference.

### FIELD OF THE INVENTION

The present invention relates to the identification of genes and proteins encoded thereof from yeast and fungi whose expression is modulated upon programmed cell death and which genes, proteins or functional fragments and equivalents thereof may be used as selective targets for drugs to treat infections caused by or associated with yeast and fungi or for the treatment of proliferative disorders or for the prevention of apoptosis in certain diseases.

### BACKGROUND OF THE INVENTION

Invasive fungal infections (e.g. Candida spp, Aspergillus spp., Fusarium spp., Zygomycetes spp.) (Walsh, 1992) have emerged during the past two decades as important pathogens causing formidable morbidity and mortality in an increasingly diverse and progressively expanding, population of immunocompromised patients. Those with the acquired immune deficiency syndrome (AIDS) constitute the most rapidly growing group of patients at risk for life-threatening mycosis. But fungal infections have also increased in frequency in several populations of other susceptible hosts, including very-low-birth-weight infants, cancer patients receiving chemotherapy, organ transplant recipients, burn patients and surgical patients with complications.

These fungal infections are not limited to humans and other mammals, but are also important in plants where they can cause diseases or cause the production of unwanted compounds (e.g. Fusarium spp., Aspergillus spp., Botritis spp., Cladosporium spp.).

Although recent advances in antifungal chemotherapy have had an impact on these mycoses, expanding populations of immunocompromised patients will require newer approaches to antifungal therapy. The discovery of novel antifungal agents is thus an essential element of any new antifungal therapy.

Classical approaches for identifying anti-fungal compounds have relied almost exclusively on inhibition of fungal or yeast growth as an endpoint. Libraries of natural products, semi-synthetic, or synthetic chemicals are screened for their ability to kill or arrest growth of the target pathogen or a related nonpathogenic model organism. These tests are cumbersome and provide no information about a compound's mechanism of action. The promising lead compounds that emerge from such screens must then be tested for possible host-toxicity and detailed mechanism of action studies must subsequently be conducted to identify the affected molecular target.

Cells from multicellular organisms can commit suicide in response to specific signals or injury by an intrinsic program of cell death. Apoptosis is a form of programmed cell death which leads to elimination of unnecessary or damaged cells. To survive, all cells from multicellular organisms depend on the constant repression of this suicide program by signals from other cells (Raff, 1992). It has been assumed that such an altruistic form of cell survival arose with multicellularity and would have been counterselected in unicellular organisms. Recent findings indicate, however, that a similar process of cell survival also operates in single-celled eukaryotes.

It has been found that expression of the mammalian Bax gene triggers cell death in Saccharomyces cerevisiae and the fission yeast Schizosaccharomyces pombe with morphological changes similar to apoptosis (Jürgensmeier et al., 1997). However, the mechanism of Bax lethality in S. cerevisiae remains unclear.

Since it has been discovered that the mammalian Bax gene triggers apoptotic changes in yeast (Ligr et al., 1998), this can be an indication that the molecular pathways eventually leading to programmed cell death may also be partially present in yeast cells and other unicellular eukaryotes.

It is an aim of the present invention to provide nucleic acid as well as polypeptide sequences which represent potential molecular targets for the identification of new compounds which can be used in alleviating diseases or conditions associated with yeast or fungi infections.

It is a further aim of the present invention to provide uses of these nucleic acid and amino acid molecules for the preparation of a medicament for treating diseases associated with yeast or fungi.

It is also an aim of the invention to provide pharmaceutical compositions and vaccines comprising these nucleic acids or polypeptides.

It is also an aim of the present invention to provide vectors comprising these nucleic acids, as well as host cells transfected or transformed with said vectors.

It is also an aim of the invention to provide antibodies against these polypeptides, which can be used as such, or in a composition as a medicine for treating diseases associated with yeast and fungi.

It is another aim of the invention to provide methods to selectively identify compounds capable of inhibiting or activating expression of such polypeptides in yeast or fungi infections. The nucleic acid and polypeptide molecules alternatively can be incorporated into an assay or kit to identify these compounds.

It is also an aim of the invention to provide a method of preventing infection with yeast or fungi.

It is also an aim of the invention to provide probes and primers derived from the nucleic acid sequences of the invention.

All the aims of the present invention have been met by the embodiments as set out below.

### SUMMARY OF THE INVENTION

The present inventors identified a range of specific nucleotide sequences which are involved in the molecular pathways eventually leading to programmed cell death. The present inventors were able to identify via macro array screening a range of genes involved in a pathway eventually leading to programmed cell death in the yeast Saccharomyces cerevisiae. As explained in Example 2, genes showing a difference of a factor 5 or more in expression as a result of Bax-induced cell death, were identified as differentially expressed candidate genes. Some of these genes are clearly down-regulated in a Bax-expressing strain, while other genes show an upregulated expression. Example 3 describes a further experiment wherein the results of differential expression were analysed using the Pathways™ software and differentially expressed nucleic acid sequence were identified.

### DETAILED DESCRIPTION OF THE INVENTION

According to a first embodiment, the invention relates to the use of a nucleic acid molecule encoding a polypeptide which is involved in a pathway eventually leading to programmed cell death of yeast or fungi and which nucleic acid sequence is selected from:
(a) a nucleic acid encoding a protein having an amino acid sequence as represented in SEQ ID NO 2, or encoding a functional equivalent, derivative or bioprecursor of said protein;
(b) a nucleic acid molecule encoding a protein having an amino acid sequence which is more than 70% similar, preferably more than 75% or 80% similar, more preferably more than 85%, 90% or 95% similar and most preferably more than 97% similar to any of the amino acid sequences shown in SEQ ID NO 2;
(c) a nucleic acid molecule encoding a protein having an amino acid sequence which is more than 70% identical, preferably more than 75% or 80% identical, more preferably more than 85%, 90% or 95% identical and most preferably more than 97% identical to any of the amino acid sequences shown in SEQ ID NO 2;
(d) a nucleic acid molecule comprising a sequence as represented in SEQ ID NO 1;
(e) a nucleic acid sequence which is more than 70% identical, preferably more than 75 or 80% identical, more preferably more than 85%, or 90% or 95% identical and most preferably more than 97% identical to any of the nucleic acid sequences shown in SEQ ID NO 1,
(f) a nucleic acid sequence encoding a functional fragment of any of the nucleic acid sequences as specified in a) to e); and
(g) the complement of any of the nucleic acid sequences as specified in a) to f), for the preparation of a medicament for treating diseases associated with yeast or fungi.

Sequence similarity searches were performed using the BLAST software package version 2. Identity and similarity percentages were calculated using BLOSUM62 as a scoring matrix.

As known in the art, “similarity” between two polypeptides is determined by comparing the amino acid sequence and its conserved amino acid substitutes of one polypeptide to the sequence of a second polypeptide. Moreover, also known in the art is “identity” which means the degree of sequence relatedness between two polypeptide or two polynucleotide sequences as determined by the identity of the match between two strings of such sequences. Both identity and similarity can be readily calculated. While there exist a number of methods to measure identity and similarity between two polynucleotide or polypeptide sequences, the terms “identity” and “similarity” are well known to skilled artisans (Carillo and Lipton, 1988). Methods commonly employed to determine identity or similarity between two sequences include, but are not limited to, those disclosed in “Guide to Huge Computers (Bishop, 1994) and Carillo and Lipton (1988). Preferred methods to determine identity are designed to give the largest match between the two sequences tested. Methods to determine identity and similarity are codified in computer programs. Preferred computer program methods to determine identity and similarity between two sequences include, but are not limited to, GCG program package (Devereux et al., 1984), BLASTP, BLASTN and FASTA (Altschul et al, 1990).

The nucleic acid sequences to be used according to this aspect of the invention from Saccharomyces cerevisiae are defined in SEQ ID NO 1.

The invention also relates to nucleic acid sequences from Candida albicans.

The expression “a pathway eventually leading to programmed cell death” refers to a sequence of steps ultimately leading to cell death and which can be triggered at various steps in this pathway by various agents, such as Bax, Bak, CED4, hydrogen peroxide, diamide and farnesol.

The yeast or fungi according to the invention may be, but are not restricted to, pathogenic yeast or fungi. As such, yeast or fungi may cause infections in healthy individuals as well as in immunocompromised patients.

The expression “treating diseases associated with yeast and fungi” not only refers to diseases or infections caused by said organisms but also refers to allergic reactions caused by said organisms, such as the so-called “professional diseases” in, for instance, bakery and brewery and that are caused by yeast or fungi which are commonly known as “non-pathogenic”.

The invention further relates to the use of nucleic acid sequence homologues of SEQ ID NO 1 but isolated from other yeast and fungi strains which are also involved in a pathway eventually leading to programmed cell death.

According to the invention, these sequences and their homologues in other yeast and fungi as well as the polypeptides which they encode represent novel molecular targets which can be incorporated into an assay to selectively identify compounds capable of inhibiting or activating expression of such polypeptides. Furthermore, the invention also relates to the potential use of said sequences in alleviating diseases or conditions associated with yeast or fungi infections, such as diseases caused by Candida spp., Aspergillus spp., Microsporum spp., Trichophyton spp., Fusarium spp., Zygomycetes spp., Botritis spp., Cladosporium spp., Malassezia spp., Epidermophyton floccosum, Blastomyces dermatitidis, Coccidioides immitis, Histoplasma capsulatum, Paracoccidioides brasiliensis, Cryptococcus neoformans, and Sporothrix schenckii.

According to a more specific embodiment, these nucleic acid sequences are derived from Saccharomyces cerevisiae, Candida albicans or Aspergillus fumigatus.

A nucleic acid sequence according to the invention may comprise an mRNA sequence or alternatively a DNA sequence and preferably a cDNA sequence. A nucleic acid sequence according to the invention may also comprise any modified nucleotide known in the art.

The present invention further relates to a nucleic acid molecule capable of selectively hybridising to at least one of the nucleic acid molecules according to the invention, or the complement thereof.

The term “selectively hybridising” or “specifically hybridising means hybridising under conditions wherein sequences can be detected which are homologues of the sequences of the invention, but which are for instance derived from heterologous cells or organisms, and wherein said sequences do not hybridize with known sequences. In a preferred embodiment, mammalian homologues can be detected. It is well known to the person skilled in the art which methods for hybridisation can be used and which conditions are necessary for selectively or specifically hybridising. Preferably, hybridization under high stringency conditions can be applied (Sambrook et al., 1989).

As such, the present invention also relates to the use of the nucleic acid sequences of the invention for detecting homologues in heterologous organisms including but not limited to mammalian organisms.

The term “nucleic acid sequence” also includes the complementary sequence to any single stranded sequence given, or the antisense version thereof.

The invention also relates to mRNA, DNA or cDNA versions of the nucleic acid molecules of the invention.

The present invention more particularly relates to an antisense molecule comprising a nucleic acid sequence capable of hybridizing to any of the above defined nucleic acid sequences.

Polynucleotides according to the invention may be inserted into vectors in an antisense orientation in order to provide for the production of antisense RNA. Antisense RNA or other antisense nucleic acids may also be produced by synthetic means.

The present invention also advantageously provides nucleic acid sequences of at least approximately 10 contiguous nucleotides of a nucleic acid according to the invention and preferably from 10 to 50 nucleotides. These sequences may, advantageously be used as probes or primers to initiate replication, or the like. Such nucleic acid sequences may be produced according to techniques well known in the art, such as by recombinant or synthetic means. The probes will hybridise specifically with any of the nucleic acid molecules of the invention. The primers will specifically amplify any of the nucleic acid molecules of the invention.

The probes or primers according to the invention may also be used in diagnostic kits or the like for detecting the presence of a nucleic acid according to the invention. These tests generally comprise contacting the probe with the sample under hybridising conditions and detecting the presence of any duplex or triplex formation between the probe and any nucleic acid in the sample.

According to the present invention these probes may be anchored to a solid support. Preferably, they are present on an array so that multiple probes can simultaneously hybridize to a single biological sample. The probes can be spotted onto the array or synthesized in situ on the array. (Lockhart et al., 1996). A single array can contain more than 100, 500 or even 1,000 different probes in discrete locations. Such arrays can be used to screen for compounds interacting with said probes.

Advantageously, the nucleic acid sequences, according to the invention may be produced using recombinant or synthetic means, such as for example using PCR cloning mechanisms which generally involve making a pair of primers, which may be from approximately 10 to 50 nucleotides to a region of the gene which is desired to be cloned, bringing the primers into contact with mRNA, cDNA, or genomic DNA from the yeast or fungal cell, performing a polymerase chain reaction under conditions which bring about amplification of the desired region, isolating the amplified region or fragment and recovering the amplified DNA. Generally, such techniques as defined herein are well known in the art, such as described in Sambrook et al. (1989). These techniques can be used to clone homologues of the nucleic acid sequences of the invention in other organisms.

The nucleic acids or oligonucleotides according to the invention may carry a revealing label. Suitable labels include radioisotopes such as ³²P, ³³P or ³⁵S, enzyme labels or other protein labels such as biotin or fluorescent markers. Such labels may be added to the nucleic acids or oligonucleotides of the invention and may be detected using techniques known in the art.

According to another embodiment of the invention, the nucleic acid sequences according to the invention as defined above may, advantageously, be included in a suitable expression vector which may be transformed, transfected or infected into a host cell. In such an expression vector the nucleic acid is operably linked to a control sequence, such as a suitable inducible promotor, or the like, to ensure expression of the proteins according to the invention in a suitable host cell. The expression vector may also comprise a reporter molecule. The expression vector may advantageously be a plasmid, cosmid, virus or other suitable vector which is known to those skilled in the art. The expression vector and the host cell defined herein also form part of the present invention. Preferably the host cell is a lower eukaryotic cell such as a yeast cell or a fungal cell. Yeast and fungal cells are particularly advantageous because they provide the necessary post-translational modifications to the expressed proteins of the invention, similar to those of the natural proteins from which they are derived. These modifications confer optimal conformation of said proteins, which when isolated may advantageously be used in kits, methods or the like.

The invention further relates to any nucleic acid as defined above for use as a medicament.

Nucleotide sequences according to the invention are particularly advantageous for providing selective therapeutic targets for treating yeast or fungi-associated infections. For example, an antisense nucleic acid capable of binding to the nucleic acid sequences according to the invention may be used to selectively inhibit expression of the corresponding polypeptides, leading to impaired growth or death of yeast and fungi with reductions of associated illnesses or diseases.

According to another embodiment, the invention also relates to the use of a polypeptide which is involved in a pathway eventually leading to programmed cell death of yeast or fungi, said polypeptide being selected from:
(a) a protein having an amino acid sequence as represented in SEQ ID NO 2, or encoding a functional equivalent, derivative or bioprecursor of said protein;
(b) a protein having an amino acid sequence which is more than 70% similar, preferably more than 75% or 80% similar, more preferably more than 85%, 90% or 95% similar and most preferably more than 97% similar to the amino acid sequence shown in SEQ ID NO 2;
(c) a protein having an amino acid sequence which is more than 70% identical, preferably more than 75% or 80% identical, more preferably more than 85%, 90% or 95% identical and most preferably more than 97% identical to the amino acid sequence shown in SEQ ID NO 2; and,
(d) a functional fragment of any of said proteins as defined in a) to c),
   for the preparation of a medicament for treating diseases associated with yeast or fungi.

The term “functional fragment” of a protein means a truncated version of the original protein or polypeptide referred to. The truncated protein sequence can vary widely in length; the minimum size being a sequence of sufficient size to provide a sequence with at least a comparable function and/or activity of the original sequence referred to, while the maximum size is not critical. In some applications, the maximum size usually is not substantially greater than that required to provide the desired activity and/or function(s) of the original sequence. A functional fragment can also relate to a subunit with similar function as said protein. Typically, the truncated amino acid sequence will range from about 5 to about 60 amino acids in length. More typically, however, the sequence will be a maximum of about 50 amino acids in length, preferably a maximum of about 60 amino acids. It is usually desirable to select sequences of at least about 10, 12 or 15 amino acids.

Functional fragments include those comprising an epitope which is specific or, unique for the proteins according to the invention. Epitopes may be determined using, for example, peptide scanning techniques as described in Geysen et al. (1996). Preferred functional fragments have a length of at least, for example, 5, 10, 25, 50, 75, 100, 125, 150, 175 or 200 amino acids.

The polypeptides to be used according to this aspect of the invention from Saccharomyces cerevisiae, are represented by SEQ ID NO 2. Also according to the invention is the use of the polypeptides from Candida albicans.

The polypeptide or protein according to the invention may also include variants of any of the polypeptides of the invention as specified above having conservative amino acid changes.

The nucleic acid molecules or the polypeptides of the invention may be provided in a pharmaceutically acceptable carrier, diluent or excipient therefor.

The present invention also relates to a vaccine for immunizing a mammal against infections caused by yeast and fungi comprising at least one (recombinant) nucleic acid molecule or at least one (recombinant) polypeptide of the invention in a pharmaceutically acceptable carrier.

Pharmaceutically acceptable carriers include any carrier that does not itself induce the production of antibodies harmful to the individual receiving the composition. Suitable carriers are typically large, slowly metabolizing macromolecules such as proteins, polysaccharides, polylactic acids, polyglycolic acids, polymeric amino acids, amino acid copolymers; and inactive virus particles. Such carriers are well known to those of ordinary skill in the art.

A “vaccine” is an immunogenic composition capable of eliciting protection against infections caused by yeast or fungi, whether partial or complete. A vaccine may also be useful for treatment of an individual, in which case it is called a therapeutic vaccine.

Said vaccine compositions may include prophylactic as well as therapeutic vaccine compositions.

The term “therapeutic” refers to a composition capable of treating infections caused by yeast or fungi.

Some of the pathways leading to apoptosis are conserved between mammalian cells and yeast or fungi. Therefore, targets which are part of such a conserved pathway may be used to stimulate or inhibit the apoptosis in mammalian cells. E.g. stimulation of apoptosis is desirable in the treatment of tumor cells/tissues.

According to another embodiment, the present invention provides a method of identifying compounds which selectively inhibit, induce or interfere with the expression/production of the polypeptides encoded by the nucleotide sequences of the invention, or compounds which selectively inhibit, activate or interfere with the functionality of polypeptides expressed from the nucleotide sequences according to the invention, or which selectively inhibit, induce or interfere with the metabolic pathways in which these polypeptides are involved. Compounds may carry agonistic or antagonistic properties. The compounds to be screened may be of extracellular, intracellular, biologic or chemical origin.

Such a screening method may comprise the following steps (a) contacting a compound to be tested with cells having a mutation which results in overexpression or underexpression of at least one of the polypeptides as defined in claim 2, in addition to contacting wild type cells with said compound, (b) monitoring the growth, death rate or activity of said mutated cells compared to said wild type cells; wherein differential growth or activity of said mutated cells is indicative of selective action of said compound on a polypeptide in the same or a parallel pathway, (c) alternatively monitoring the growth, death rate or activity of said mutated cells compared to mutated cells which were not contacted with the compound to be tested, wherein differential growth or activity of said mutated yeast of fungi cells is indicative of selective action of said compound on a polypeptide in the same or a parallel pathway, (d) alternatively monitoring changes in morphologic and/or functional properties of components in said mutated cells caused by the addition of the compound to be tested.

The term “cells” as used above relates to any type of cells such as, but not limited to bacterial, yeast, fungal, plant or human cells.

Compounds found using this approach may additionally be tested on their efficiency in killing or inhibiting the growth of wild type cells in order to confirm their utility as medicament for treating wild type pathogenic strains/tumor cells.

According to the invention, the term “mutation” includes point mutations, deletions, insertions, duplications or any modification in the nucleic acid encoding said polypeptide, or at a different location in the genome of said cells, influencing the expression of said nucleic acid or polypeptide. In case point mutations occur, the number of nucleotides will be identical compared to the original sequence; only a change in nucleotide sequence can be observed. This stands in contrast with the other listed mutations where the number of the nucleotides will be different from the number observed in the wild type sequence and consequently will also reflect in a change of the nucleotide sequence.

Changes in morphologic and/or functional properties of cell components which, can be monitored include for example morphological and molecular changes such as abnormal cell morphology, nuclear fragmentation, DNA breakage or changes in the expression of certain enzymes such as caspases, as well as monitoring changes in membrane potential or activity of mitochondria and release of cytochrome c from mitochondria. All these changes can be monitored on the whole cell which is contacted to the compound to be tested.

The invention also relates to a method of identifying compounds which selectively modulate expression or functionality of polypeptides involved in a pathway eventually leading to programmed cell death of yeast and fungi or in metabolic pathways in which said polypeptides are involved, which method comprises (a) contacting a compound to be tested with yeast or fungal cells transformed, transfected or infected with an expression vector comprising an antisense sequence of at least one of the nucleic acid sequences, which expression results in underexpression of said polypeptide, in addition to contacting one or more wild type cells with said compound, (b) monitoring the growth, death rate or activity of said transformed, transfected or infected cells compared to said wild type cells; wherein differential growth or activity of said transformed, transfected or infected yeast or fungal cells is indicative of selective action of said compound on a polypeptide in the same or a parallel pathway, (c) alternatively monitoring the growth, death rate or activity of said transformed, transfected or infected cells compared to transformed, transfected or infected cells which were not contacted with the compound to be tested, wherein differential growth or activity of said mutated yeast or fungi cells is indicative of selective action of said compound on a polypeptide in the same or a parallel pathway, (d) alternatively monitoring changes in morphologic and/or functional properties of components in said transformed, transfected or infected cells caused by the addition of the compound to be tested.

Alternative methods for identifying compounds which selectively modulate expression or functionality of polypeptides involved in a pathway eventually leading to programmed cell death of yeast or fungi or in metabolic pathways in which said compounds are involved, may comprise the use of any other method known in the art resulting in gene activation, gene inactivation, gene modulation or gene silencing.

The invention also relates to a method of identifying compounds which selectively modulate expression of polypeptides which are involved in a pathway eventually leading to programmed cell death of yeast or fungi which method comprises (a) contacting host cells transformed, transfected or infected with an expression vector comprising a promoter sequence of a nucleic acid molecule as defined in claim 1 joined in frame with a reporter gene and (b) monitoring increased or decreased expression of said reporter gene caused by the addition of the compound being tested. This enables to analyze the influence of the compound onto all/most aspects of transcriptional activation. Alternatively additional tests can routinely be performed to test the influence of the compound onto mRNA stability, translation and protein stability. All these aspects influence the concentration of corresponding proteins and consequently influence the effect of these on the metabolism of the cell.

The invention further relates to a method of identifying compounds which bind to or modulate the properties of polypeptides which are involved in a pathway eventually leading to programmed cell death of yeast or fungi, which method comprises (a) contacting a compound to be tested with at least one of the polypeptides as defined in claim 2, (b) detecting the complex formed between the compound to be tested and said polypeptide, (c) alternatively, examining the diminution/increase of complex formation between said polypeptide and a receptor/binding partner, caused by the addition of the compound being tested, (c) alternatively, examining the alteration in the functional activity of the polypeptide, caused by the addition of the compound being tested.

Detection of the complex formation can be performed using several approaches. First, binding of a compound onto a polypeptide can be studied using classical binding tests: one of the binding partners, compound or polypeptide is labeled and interaction of both is measured. Most of these tests comprise following steps: incubating both binding partners in conditions where binding is allowed, separation of free label from bound label present in the complex formed between both partners, and measuring the number of labeled complexes formed. Separation of free and bound label can be performed via filtration, centrifugation or other means as known by the person skilled in the art. Other techniques allow visualization of complex formation without the need of such a separating step. For example, test systems using SPA (scintillation proximity assay) beads are based on the principle that radioactive ³H can only be measured when present in scintillation fluid. SPA beads contain scintillation fluid and can be coated with one of the binding partners. When this bead is approached and binds the other binding partner which is radioactively labeled, a signal will be detected allowing the complex to be visualized. Binding of the radioactive compound onto the scintillation bead is needed in order to result in a detectable signal; non-bound radioactive partners that stay free into the solution will not result in a detectable signal.

The protein or peptide fragments according to the invention employed in such a method may be for example in solution or coated on suspended beads as described above. Alternatively, these can be affixed to a solid support, borne on a cell or phage surface or located intracellularly.

When protein or peptide fragments are coated on solid supports, they can be tested for their binding affinity for large numbers of compounds. These can be used in different kinds of high throughput screenings in order to identify compounds having suitable binding affinity to the polypeptides according to the invention. Platform technologies or technologies based on SPR (see below) can be applied.

One may measure for example, the formation of complexes between the proteins of the invention and the compound being tested. Alternatively, one may examine the diminution or increase of complex formation between the protein according to the invention and a receptor/binding partner caused by the compound being tested.

Proteins which interact with the polypeptide of the invention may be identified by investigating protein-protein interactions using the two-hybrid vector system first proposed by Chien et al. (1991).

This technique is based on functional reconstitution in vivo of a transcription factor which activates a reporter gene. More particularly the technique comprises providing an appropriate host cell with a DNA construct comprising a reporter gene under the control of a promoter regulated by a transcription factor having a DNA binding domain and an activating domain, expressing in the host cell a first hybrid DNA sequence encoding a first fusion of a fragment or all of a nucleic acid sequence according to the invention and either said DNA binding domain or said activating domain of the transcription factor, expressing in the host at least one second hybrid DNA sequence, such as a library or the like, encoding putative binding proteins to be investigated together with the DNA binding or activating domain of the transcription factor which is not incorporated in the first fusion; detecting any binding of the proteins to be investigated with a protein according to the invention by detecting for the presence of any reporter gene product in the host cell; optionally isolating second hybrid DNA sequences encoding the binding protein.

An example of such a technique utilizes the GAL4 protein in yeast. GAL4 is a transcriptional activator of galactose metabolism in yeast and has a separate domain for binding to activators upstream of the galactose metabolizing genes as well as a protein-binding domain. Nucleotide vectors may be constructed, one of which comprises the nucleotide residues encoding the DNA binding domain of GAL4. These binding domain residues may be fused to a known protein encoding sequence, such as for example the nucleic acids according to the invention. The other vector comprises the residues encoding the protein-binding domain of GAL4. These residues are fused to residues encoding a test protein. Any interaction between polypeptides encoded by the nucleic acid according to the invention and the protein to be tested leads to transcriptional activation of a reporter molecule in a GAL-4 transcription deficient yeast cell into which the vectors have been transformed. Preferably, a reporter molecule such as β-galactosidase is activated upon restoration of transcription of the yeast galactose metabolism genes. Alternatively, other reporter proteins can be used such as EGFP (enhanced green fluorescent protein), or hEGFP. This latter has a decreased lifetime enabling the system to screen for compounds improving the interaction of studied binding partners.

The two-hybrid approach was first developed for yeast, and is an ideal screening system when looking for compounds active in killing yeast or fungi. Indeed, proteins expressed in this system will most probably carry the correct modifications as found in the pathogenic yeast strains. In addition, compounds active in this test system allow to screen and select compounds which are able to enter the cell, this selection is not possible when using in vitro test systems. When compounds are needed to target mammalian cells, modification of the studied proteins can be different, changing the structure of corresponding proteins. Moreover working with yeast might block certain compounds to enter the cell, which are normally able to traverse the mammalian cell membrane. Consequently, working with mammalian two-hybrid system for this purpose will give already an immediate selection of the compounds that may enter mammalian cells.

Alternative in vitro methods can be used to investigate protein-protein interactions. Protein interaction analysis in vitro can shed light on their role in the intact cell by providing valuable information on specificity, affinity, and structure-function relation ship. Significant process in this respect has become with the advent, in the last few years, of commercially available biosensor technology. This allows to study macromolecular interactions in real-time, providing a wealth of high-quality data that can be used for kinetic analysis, affinity measurements, competition studies, etc. A major advantage of biosensor analysis is that there is no requirement for labeling one of the interacting components and then separating bound from free molecules—a fact that simplifies experimental procedures and provides more accurate measurements. The principle of surface plasmon resonance (SPR) is based on the detection of a change of the refractive index of the medium when a compound or protein binds to an immobilized partner molecule. For the SPR technology, one needs to load one of the interacting partners to the chip surface, followed by the superfusion of the second binding partner or more molecules. The second partner can be available as purified product, but alternatively a complex suspension containing this partner can also be used. Interaction of two or more compounds can be analyzed, alternatively, compounds can be identified interfering or increasing this binding affinity towards each other.

SPR is not restricted to protein-protein interactions; any macromolecule with a suitable size will change the refractive index of the medium in contact with the biosensor surface and therefore give a signal. Studies have been done with protein-DNA interactions, as well as protein-lipid interactions. Moreover intact viruses, and even cells, can also be injected over the biosensor surface, in order to analyze their binding to receptors, lectins, and so on.

Alternatively, NMR is also an excellent tool for a detailed study of protein-protein or DNA-protein interactions. Isotope edited or isotope filtered experiments whereby one compound is isotopically labeled with 15N or 13C are an ideal way to study these complexes. This method does not allow high throughput analysis of compounds interfering or enhancing molecular interactions. Nevertheless, medium or low throughput systems can be used to confirm results obtained by the high throughout assays or in cases where none of the binding partners are labeled. Other techniques which can be used to study interactions are: overlay, ligand blotting, band-shift, co-immuno-precipitation, size exclusion chromatography and microcalorimetry (In. “Protein targeting Protocols” Ed. Clegg R. A. Humana Press, Totowa, N.J.).

Compounds modulating pathways leading to apoptosis may change the activity of the polypeptide of the invention. Therefore screening tests may be setup looking for altered protein activity of the polypeptide of the invention. Based on the amino acid sequence a possible function of the polypeptide might be envisaged; activities can be confirmed and corresponding activity test can be started.

Alternatively additional tests can be performed to test the influence of the compound onto protein stability, post-translational modification, precursor processing and protein translocation. All these aspects influence the concentration and/or activity of corresponding proteins and consequently influence the effect of these onto the metabolism of the cell. Also here, medium or low throughput systems can be used to confirm results obtained by the high throughout assays.

In cases compounds need to be found to target tumor cells, screening assays will have to be used focused on the stimulation of the apoptotic pathway. This invention therefore also relates to in vitro and in vivo model systems comprising tumor tissue or, cells expressing the polypeptides according to the invention which can be used to screen for therapeutic agents. In vivo model systems allow to test for compound efficacy but also the toxicity of these compounds can be tested. The compounds identified using any of the methods described in the invention not only include compounds which exert their effect in promoting cell death of yeast and fungi, but also include compounds which prevent or delay cell death. The latter compounds can be used to prevent or delay apoptosis of endogenic yeast or fungi in humans and other mammals which may be caused by pathogens or toxic environmental components.

According to a preferred aspect of the invention, the yeast or fungi according to any of the methods described, are chosen from Saccharomyces cerevisiae, Schizosaccharomyces pombe, Candida albicans, or Aspergillus fumigatus.

The invention also relates to a compound identified using any of the methods of the invention.

Compounds identifiable or identified using a method according to the invention, may advantageously be used as a medicament. The invention also relates to a method for treating diseases associated with yeast or fungi comprising admixing a compound obtainable by a method of the invention with a suitable pharmaceutically acceptable carrier.

The compounds of the invention can be used for the preparation of a medicament to treat diseases or conditions associated with yeast and fungi infections, for instance Candida spp., Aspergillus spp., Microsporum spp., Trichophyton spp., Fusarium spp., Zygomycetes spp., Botritis, spp., Cladosporium spp., Malassezia spp., Epidermophyton floccosum, Blastomyces dermatitidis, Coccidioides immitis, Histoplasma capsulatum, Paracoccidioides brasiliensis, Cryptococcus neoformans, and Sporothrix schenckii infections.

These compounds may also advantageously be included in a pharmaceutical composition together with a pharmaceutically acceptable carrier, diluent or excipient therefor.

A medicament according to the invention not only relates to fungistatic compounds for treating humans or mammals but also relates to fungicides for treating plants.

The invention also relates to genetically modified yeast or fungi in which modification results in the overexpression or underexpression of at least one of the nucleic acids or polypeptides of the invention, which overexpression or underexpression of said nucleic acid or polypeptide prevents or delays apoptosis of said genetically modified yeast or fungi. These genetically modified organisms may have a positive effect on the endogenic flora of humans and other mammals. The genetically modified yeast or fungi can be included in a pharmaceutical composition or can be used for the preparation of a medicament for prophylactic or therapeutic use.

Also according to the invention is the use of a compound obtainable by a method of the invention for the preparation of a medicament for modifying the endogenic flora of humans and other mammals.

According to the invention, the polypeptide as defined above may be used as a medicament.

Also encompassed within the present invention are antibodies, monoclonal or polyclonal, capable of specifically binding to one or more epitopes of the proteins of the invention. The term “specific binding” implies that there is substantially no cross-reaction of the antibody with other proteins.

The antibodies according to the invention may be produced according to techniques which are known to those skilled in the art. Monoclonal antibodies may be prepared using conventional hybridoma technology as described by Kohler and Milstein (1979). Polyclonal antibodies may also be prepared using conventional technology well known to those skilled in the art, and which comprises inoculating a host animal, such as a mouse, with a protein or epitope according to the invention and recovering the immune serum. The present invention also includes fragments of whole antibodies which maintain their binding activity, such as for example, Fv, F(ab′) and F(ab′)₂ fragments as well as single chain antibodies.

Antibodies according to the invention may also be used in a method of detecting the presence of a polypeptide according to the invention, which method comprises reacting the antibody with a sample and identifying any protein bound to said antibody. A kit may also be provided for performing said method which comprises an antibody according to the invention and means for reacting the antibody with said sample.

The antibodies according to the invention may be used as a medicament or may be comprised in a pharmaceutical composition. According to a more specific embodiment, the antibodies may be used in the preparation of a medicament for treating diseases associated with yeast and fungi such as, but not restricted to, Candida albicans, Aspergillus spp., Fusarium spp., Botritis, spp., Cladosporium spp.

The invention also relates to a method of preventing infection with yeast or fungi, comprising administering a composition containing at least one polypeptide of the invention to a mammal in effective amount to stimulate the production of protective, antibody or protective T-cell response.

According to another embodiment, the invention relates to a genetically modified mammalian cell or non-human organism in which modification results in the overexpression or underexpression of at least one of the nucleic acids of the invention or a human homologue thereof or at least one of the polypeptides of the invention or a human homologue thereof, which overexpression or underexpression of said nucleic acid or polypeptide prevents or delays apoptosis of said genetically modified mammalian cell or in said genetically modified non-human organism.

Human homologues according to the invention can be obtained by selective hybridisation of the yeast and candida nucleic acid molecules of the invention against human genome or cDNA libraries according to methods well known in the art (Sambrook et al., 1989). Human polypeptide homologues are obtained from the corresponding human nucleic acid homologous nucleotide sequences.

The invention also relates to a method for identifying compounds for stimulating or inhibiting apoptosis comprising the use of at least one of the nucleic acid sequences of the invention or a human homologue thereof and/or at least one of the polypeptides of the invention or a human homologue thereof and/or a genetically modified mammalian cell or non-human organism as described in the invention.

The invention further relates to the compounds identifiable according to the above-described method and its use as a medicament.

The invention further relates to a method for preparing a pharmaceutical composition for treating proliferative disorders or for preventing apoptosis in certain diseases comprising admixing a compound of this invention with a suitable pharmaceutically acceptable carrier.

The expression “proliferative disorders” or “proliferative diseases” refers to an abnormality within a patient or animal such as cancer. Normal cells start to proliferate due to a change in the coding or non-coding sequence of the DNA resulting in a swollen or distended tissue. Mutation may arise without obvious cause. An abnormal benign or malignant mass of tissue is formed that is not inflammatory. Cells of pre-existent tissue start to divide unexpectedly and resulting cell mass possesses no physiologic function.

The expression “apoptosis” or “apoptosis-related diseases” includes diseases such as autoimmunity diseases, ischemia, diseases related with viral infections or neurodegenerations.

The invention also relates to the use of compounds obtainable by the above described methods for the preparation of a medicament for treating proliferative disorders or for preventing apoptosis in certain disorders.

According to another embodiment, the invention relates to the use of a nucleic acid molecule or a polypeptide described in the invention or human homologues thereof for treating proliferative disorders or for the prevention of apoptosis in certain diseases.

The invention also relates to a pharmaceutical composition for use as a medicament for treating proliferative disorders or for the prevention of apoptosis in certain diseases comprising a nucleic acid molecule of the invention or a human homologue thereof or a polypeptide of the invention or a human homologue thereof together with a pharmaceutically acceptable carrier diluent or excipient therefor.

The invention also relates to a vaccine for immunizing mammals against proliferative disorders or for preventing apoptosis in certain diseases comprising least one nucleic acid molecule of the invention or a human homologue thereof or at least one polypeptide of the invention or a human analogue thereof in a pharmaceutically acceptable carrier.

The invention also relates to the use of an antibody of the invention capable of binding to at least one of the polypeptides of the invention or a human homologue thereof for the preparation of a medicament for treating proliferative disorders or for the prevention of apoptosis in certain diseases.

According to yet another embodiment, the invention relates to an expression vector comprising a human homologue of a nucleic acid sequence of the invention. Said expression vector may comprise an inducible promoter and may further comprise a sequence encoding a reporter molecule.

The invention also relates to a host cell transformed, transfected or infected with any of the above described vectors.

According to another embodiment the invention relates to a nucleic acid molecule comprising a human homologue of at least one of the nucleic acid sequences described in the invention.

The invention also relates to an antisense molecule comprising a nucleic acid sequence capable of selectively hybridising to a nucleic acid molecule which is a human analogue of the invention.

The invention also relates to a polypeptide encoded by the nucleic acid molecule comprising said human homologues of the nucleic acids described in the invention.

The invention, now being generally described, may be more clearly understood by reference to the following examples, which are included merely for purposes of illustration of certain aspects and embodiments of the present invention and are not intended to limit the invention. The contents of all references referred to in this text are hereby incorporated by reference.

### EXAMPLES

### Example 1

### Differential Gene Expression Analysis Upon Bax-Induced Cell Death

Materials and Media

Bacterial strain Escherichia coli MC1061 (Casadaban and Cohen, 1980) was used for the construction and the amplification of plasmids. Yeast strains were grown under normal conditions on standard media (Sherman et al., 1979). The Saccharomyces cereviseae strain INVSc1 (Invitrogen®) was transformed by means of the lithium acetate method (Schiestl and Gietz, 1989) with YIpUTyL or_YIpUTYLMuBax, after linearisation in the Ty δ element (Zhu, 1986).

Cloning of Mouse BAX cDNA

Mouse Bax cDNA, encoding the mouse Bax-α protein, was cloned by Pfu DNA polymerase (Stratagene®) chain reaction amplification (PCR) from an EL4/13.18 thymoma cDNA library (BCCM™/LMBP-LIB15) by making use of the primers:
5′-ATGGACGGGTCCGGGAGCAG-3′ and
5′-TCAGCCCATCTTCTTCCAGATGGTGAG-3′.

The resulting PCR product was cloned in a HindII-opened pUC19 according to standard procedures (Sambrook J. et al., 1989).

Plasmid Constructions

The 2μ ori and the URA3 marker gene were removed from pUT332 (Gatignol et al., 1990) by successive digestions with ClaI and BglII. A BamHI-HindIII GALL promoter fragment was ligated into the BglII-HindIII-opened plasmid._A XbaI-FspI FLP terminator fragment was inserted into this XbaI-HindIII(blunted)-opened plasmid so that the plasmid YIpUT was obtained. Insertion of a blunted EcoRI-BsaAI Ty δ element in the KpnI-AatII-opened and blunted YIpUT resulted in the plasmid YIpUTy. Subsequent insertion of the LEU2 marker gene, as a blunted BsaAI-BsrGI fragment, in the BamHI-openend and blunted YIpUTy resulted in the plasmid YIpUTyL.

Mouse Bax cDNA was excised from pUC19 by digestion with XbaI and HindIII and subcloned into the XbaI-HindIII-opened plasmid YIpUTyL, obtaining the final expression plasmid YIpUTyLMuBax.

The plasmid YIpUTyLMuBax has been deposited in the BCCM™/LMBP culture collection as p5CTyGALmBax with accession number 3871 under restricted use.

GeneFilters

The Yeast GeneFilters™ were purchased from Research Genetics Inc. (Huntsville, Ala., USA).

The Yeast GeneFilters™ are hybridization ready nylon membranes containing a total of 6144 gene ORFs (Open Reading Frames) individually amplified by PCR and spotted on 2 nylon membrane filters (Filter I and II). The filters are cut in the upper right corner and the DNA is on the labeled side of the filter.

Filter I contains 3072 ORFs organized into two fields (fields 1 and 2). Each field contains 1536 ORFs divided into 8 grids (A, B, C, D, E, F, G and H). The grids are organized in 24 rows and 8 columns.

Filter II contains 3072 ORFs organized in two fields (field 3 and 4). Fields 3 and 4 are organized in the same way as fields 1 and 2.

The Yeast ORF Target

The yeast filters consist of over 6000 PCR products corresponding to 6144 yeast ORFs derived from the SGD. The PCR reactions used ORF specific primer pairs designed to amplify the entire open reading frame. The primers were generated from unique sequences containing the start codon ATG and termination codon (kindly provided by M. Cherry at Stanford Genome Center). Thus the PCR product contains the complete open reading frame including the start and stop codons. These products were purified and resuspended at 50 nanograms per microliter in a colored solution to allow the printing to be monitored. A robotic device was used to spot approximately 1/10 of a microliter of the denatured PCR product solution on a positively charged nylon membrane. The DNA was then UV cross-linked to the membrane.

Results

Induction of Bax-Expression in Yeast Cells

S. cerevsiae cells (strain INVSc1) were transformed with the expression plasmid YIpUTyLMuBax or the parental plasmid YIpUTyL as a negative control. Alternative yeast strains (such as W303-1A (Thomas and Rothstein, 1989)) with equivalent properties are known in the art and can also be used.

The Ty δ element of both plasmids allowed a stable multi-copy integration in the genome of the yeast cell. Southern analysis of the cells containing YIpUTyLMuBax revealed the integration of 5 GAL1-controlled Bax-cassettes near Ty elements.

The yeast cells containing YIpUTyLMuBax and the yeast cells containing YIpUTyL were grown overnight in in 10 ml minimal glucose-containing medium. The precultures were then further diluted to an OD₆₀₀ of 0.2 in 100 ml minimal glucose-containing medium and grown until an OD₆₀₀ of 1 was reached. Subsequently, the yeast cells containing YIpUTyL were washed and a dilution thereof was transferred into, 100 ml galactose-containing medium and incubated for 15 hours. After this additional period the cultures reached an OD₆₀₀ of 1. The yeast cells containing YipUTyLMuBax were also washed and transferred into 100 ml galactose-containing medium and incubated for 15 hours.

RNA Isolation

Total RNA was isolated using RNApure™ Reagent (Genhunter Corporation Nashville, Tenn., USA) according to the GenHunter protocol. 1.5 10⁹ cells were concentrated in a microcentrifuge tube and 1 ml RNApure™ Reagent was added together with 1 g of glass pearls. The yeast cells were broken by thorough mixing during five 2-minutes periods, and placed on ice in-between to avoid RNA digestion. Chloroform (150 μl) was added to the lysate and centrifuged for 10 min at 4° C. and at 15000 rpm. The supernatant was transferred to a new tube and the RNA was precipitated with an equal volume of isopropanol. After 10 min incubation on ice, the RNA was pelleted by centrifugation and the pellet was washed with 70% ice-cold ethanol. The dried RNA pellet was resuspended in 50 μl RNAse free dH₂O.

First Strand cDNA Synthesis in the Presence of α-³³P dCTP

Probes with high specific activity were prepared by first strand cDNA synthesis using total RNA isolated from INVSc1 YIpUTyLMuBax or INVSc1 YIpUTyL yeast cells and incorporation of α-³³P dCTP as follows: 2 μl (1 μg/ml) of Oligo dT was added to 20 μg of total RNA in a maximal volume of 8 μl RNase-free dH₂O and incubated at 70° C. for 10 min. After cooling down on ice for 1 min, the following components were added:
6 μl 5× concentrated First Strand Buffer (GIBCO-BRL)
1 μl 0.1 M DTT
1 μl RNase Block (40 units/μl) (Stratagene)
1.5 μl 20 mM dXTP-solution (X=A, G and T) (Pharmacia)
1.5 μl SuperScript™ Reverse Transcriptase (200 units/μl) (GIBCO-BRL)
10 μl α-³³P dCTP (10 mCi/ml, 3000 Ci/mmol) (Amersham)
and incubated for 2 h at 37° C. during which first strand cDNA synthesis took place.
   Unincorporated label was separated from the probe on a Sephadex G-50 column (Pharmacia). The radioactivity incorporated in the probe was measured by liquid scintillation. The specific activity of the probes was 3 or 5 10⁸ cpm/μg for both the INVSc1YIpUTyL and the INVSc1YIpUTyLMuBax probe.

Additionally, the length of first strand cDNA probes was controlled on an alkaline 2% agarose gel using standard electrophoresis techniques, and resulted in the detection, via stimulated phosphorescence autoradiography, of the bulk of the fragments around 500 bp.

Hybridisation with the S. cerevisiae Yeast GeneFilters™ and Signal Detection

The Yeast GeneFilters™ were successively hybridised with the α-³³P dCTP labelled cDNA probes using the MicroHyb™ solution provided by the manufacturer (Research Genetics Inc., Huntsville, Ala., USA). This solution was applied as well in the prehybridisation step as during hybridisation. The MicroHyb solution contains formamide to allow hybridisation to occur at lower temperatures.

The hybridisation experiment was performed essentially as follows: during prehybridisation, the Yeast GeneFilters™ were placed in a hybridisation flask (35×250 mm) filled with 5 or 10 ml MicroHyb™ solution (42° C.) containing 5 PI polydA (0, 5 or 1 μg/ml) and incubated for 24 hours at 42° C. whilst rotating (10 rpm). After disposal of the prehybridisation solution, the denatured (3 min at 100° C.) cDNA was added in 5 ml prewarmed MicroHyb solution and again incubated overnight at 42° C. whilst rotating. Following two wash steps of 20 min in wash buffer (2×SSC, 1% SDS) at 50° C., a third wash step was performed in a second wash buffer (0.5×SSC, 1% SDS) for an additional 15 min at room temperature. The Yeast GeneFilters™ were placed in a PhosphorImager™ cassette with storage phosphorscreen. After 4 days of development the screen was developed and scanned using the PhosphorImager™ 455 SI from Molecular Dynamics.

In-between the hybridisation experiments, the filters were stripped off by incubation in 500 ml of a 0.5% SDS solution (prewarmed to near boiling temperature) during at least 1 hour at room temperature.

### Example 2

### Quantification of Hybridisation Signals

Quantification of the hybridisation signals was done using the ImageQuant™ 4.1 software tool from Molecular Dynamics (Sunnyvale, Calif.). The quantification was performed per grid of the Yeast GeneFilters™, and by drawing a roster of 24 columns and 8 rows onto each grid of each filter. As such, each rectangle of the roster corresponds to a spot on the Yeast GeneFilters™. Subsequently, from each grid a volume-report (quantification) was drawn up and the data were transferred to a Microsoft™ Excel sheet. Also for each grid a correction factor was calculated. Signals neighboring big and dark spots were separately quantified. For each grid, a background level was calculated.

Statistical Processing of Quantification Results

The statistical processing of the results was accomplished in Microsoft™ Excel. For each grid, the following statistical functions were separately defined:

1. The frequency of occurrence of the values in a precisely defined intensity range (data range), established between 1000 and 61000, split up in intervals of 5000.

2. The frequency in terms of percentage.

3. The cumulated frequency in terms of percentage.

These numeric values were used for the graphical display of the cumulated frequency in terms of percentage.

Subsequently, the results of the two experiments (hybridization with cDNA from YIpUTyL containing INVSc1 cells and hybridization with cDNA from YIpUTyLMuBax containing INVSc1 cells) were integrated by determination of a second range of statistical functions:
1. The average of the values of the two experiments for each spot on the filter.
2. The standard deviation on this average. This is a measure for the distribution of the values around this average.
3. The standard deviation in terms of percentage.

The quotient of the values of the second experiment (Bax expression) against the values from the first experiment (control) was determined. This immediately ave the factor by which the expression of a specific gene is changed upon Bax induction.

In order to process all these data and to be able to discriminate between differences in gene expression, a gene showing a standard deviation in terms of percentage of at least 90% and a difference of a factor 5 in expression as a result of Bax induction, was identified as a differentially expressed candidate gene. Requantification of these candidates confirmed their selection.

When the expression pattern of 6144 genes is compared in the two experiments, it could be concluded that the expression profile of 142 genes (this is 2.3%) has been changed with at least of factor 5. An overview of these genes as well as the factors with which they are up- or down-regulated was obtained. The sequences of the genes and the amino acid sequences which they encode are in SEQ ID No 1 and, SEQ ID No 2, respectively.

Regarding genes modulated by Bax expression in S. cerevisiae; we include all the genes for which mRNA levels changed more than fivefold in a first experiment. The factor by which the transcript level was affected, is expressed as the Qt value. A Qt value higher than 1 indicates upregulation while a Qt value lower than 1 indicates a downregulation. For instance, a Qt of 0.5 indicates a two-fold lower transcript level of a particular mRNA due to Bax expression in S. cerevisiae. Upregulation or downregulation of a specific mRNA is stated when Qt had a value of at least five or at most 0.21, respectively.

### Example 3

### Quantification of Hybridisation Signals Using the Pathways™ Software

Quantification of the hybridisation signals was done using the Pathways™ Software (Research Genetics) and these signals were normalised against all data points. Comparison of these normalised data revealed differentially expressed candidate genes. Visual inspection of the hybridisation spots confirmed their selection. An overview of these genes as well as the factors with which they are up- or down-regulated is as follows. Genes are modulated by Bax expression in S. cerevisiae. This includes the gene for which mRNA levels changed significantly in a second experiment; the Qt values were calculated using the Pathways Software (Research Genetics).

| ORF | Qt | Sequence ID Number |
|---|---|---|
| YGR183C | 0.781 | SEQ ID NO 1 |

This is also known as QCR9.

Surprisingly, using this Software package for analysing the results in this example and when compared to the results of example 2, some additional genes were found which expression are up- or down-regulated upon Bax expression in S. cerevisiae.

### Example 4

### Search for Homologues in Candida albicans

Sequence similarity searches against public and commercial sequence databases were performed with the BLAST software package (Altschul et al., 1990) version 2. Both the original nucleotide sequence and the six-frame conceptual translations were used as query sequences. The used public databases were the EMBL nucleotide sequence database (Stoesser et al., 1998), the SWISS-PROT protein sequence database and its supplement TrEMBL (Bairoch and Apweiler, 1998), and the ALCES Candida albicans sequence database (Stanford University, University of Minesota). The commercial sequence database used was the PathoSeq™ microbial genomic database (Incyte Pharmaceuticals Inc., Palo Alto, Calif., USA).

Sequence similarity searches were performed using the BLAST software package version 2. The identity between 2 sequences was calculated as percentage identical residues, the similarity percentage between two sequences was calculated using BLOSUM62 as a scoring matrix.

### Example 5

### Screening for Compounds Modulating Expression of Polypeptides Involved in Induction of Cell Death of C. albicans

The method proposed is based on observations (Sandbaken et al., 1990; Hinnebusch and Liebman 1991; Ribogene PCT WO 95/11969, 1995) suggesting that underexpression or overexpression of any component of a process (e.g. translation) could lead to altered sensitivity to an inhibitor of a relevant step in that process. Such an inhibitor should be more potent against a cell limited by a deficiency in the macromolecule catalyzing that step and/or less potent macromolecule, as compared to the wild type (WT) cell.

Mutant yeast strains, for example, have shown that some steps of translation are sensitive to the stoichiometry of macromolecules involved. (Sandbaken et al., 1990). Such strains are more sensitive to compounds which specifically perturb translation (by acting on a component that participates in translation) but are equally sensitive to compounds with other mechanisms of action.

This method thus not only provides a means to identify whether a test compound perturbs a certain process but also an indication of the site at which it exerts its effect. The component which is present in altered form or amount in a cell whose growth is affected by a test compound is potentially the site of action of the test compound.

The assay to be set up involves measurement of growth or death rate of an isogenic strain which has been modified only in a certain specific allele, relative to a wild type (WT) Candida albicans strain, in the presence of R-compounds. Strains can be ones in which the expression of a specific protein is impaired upon induction of anti-sense or strains which carry disruptions in an essential gene. An in silico approach to find novel genes in Candida albicans will be performed. A number of essential genes identified in this way will be disrupted (in one allele) and the resulting strains can be used for comparative growth and/or death rate screening.

### Example 6

### Assay for High Throughput Screening for Drugs

35 μl minimal medium (S medium+2% galactose+2% maltose) is transferred in a transparent flat-bottomed 96 well plate (MW96) using an automated pipetting system (Multidrop, Labsystems). A 96-channel pipettor (Hydra, Robbins Scientific) transfers 2.5 μl of R-compound at 10⁻³ M in DMSO from a stock plate into the assay plate.

The selected Candida albicans strains (mutant and parent (CAI-4) strain) are stored as glycerol stocks (15%) at −70° C. The strains are streaked out on selective plates (SD medium) and incubated for two days at 30° C. For the parent strain, CAI-4, the medium is always supplemented with 20 μg/ml uridine. A single colony is scooped up and resuspended in 1 ml minimal medium (S medium+2% galactose+2% maltose). Cells are incubated at 30° C. for 8 hours while shaking at 250 rpm. A 10 ml culture is inoculated at 250.000 cells/ml. Cultures are incubated at 30° C. for 24 hours while shaking at 250 rpm. Cells are counted in Coulter counter and the final culture (S medium+2% galactose+2% maltose) is inoculated at 20.000 to 50.000 cells/ml. Cultures are grown at 30° C. while shaking at 250 rpm until a final OD₆₀₀ of 0.24 (+/−0.04) is reached.

200 μl of this yeast suspension is added to all wells of MW96 plates containing R-compounds in a 450 μl total volume. MW96 plates are incubated (static) at 30° C. for 48 hours.

Optical densities are measured after 48 hours.

Test growth is expressed as a percentage of positive control growth for both mutant (x) and wild type (y) strains. The ratio (x/y) of these derived variables is calculated.

### REFERENCES

Altschul, S. F. Gish, W., Miller, W., Myers, E. W. and Lipman, D. J. (1990). Basic local alignment search tool. J. Mol. Biol. 215, 403-410.
Bairoch, A. and Apweiler, R. (1998). The SWISS-PROT protein sequence data bank and its supplement TrEMBL in 1998. Nucleic Acids Res. 26, 38-42.
Bishop, M. J., ed. (1994). Guide to Huge Computers, Academic Press, San Diego.
Carillo, H. and Lipton, D. (1988). SIAM J. Applied Math. 48, 1073.
Casadaban, M. J., and Cohen, S. N. (1980). Analysis of gene control signals by DNA fusion and cloning in Escherichia coli. J. Mol. Biol. 138, 179-207.
Chien, C. T., Bartel, P. L., Sternglanz, R., and Fields, S. (1991). The 2-hybrid system—a method to identify and clone genes for proteins that interact with a protein of interest. Proc. Natl. Acad Sci USA 88, 9578-9582.
Devereux, J., Haeberli, P. and Smithies, O. (1984). A comprehensive set of sequence analysis programs for the VAX. Nucleic Acids Research 12, 387-395.
Gatignol, A., Dassin, M. and Tiraby, G. (1990). Cloning of Saccharomyces cerevisiae promoters using a probe vector based on phleomycin resistance. Gene 91, 35-41.
Geysen, H. M., Rodda, S. J. and Mason, T. J. (1986). A priori delineation of a peptide which mimics a discontinuous antigenic determinant. Mol. Immunol. 23, 709-715.
Hinnebush, A. G. and Liebman, S. W., in: The Molecular Biology of the Yeast Saccharomyces (1991). Broach, J. R., Pringle, J. R. and Jones, E. W., eds., CSH Laboratory Press, NY.
Jürgenmeiser, J. M., Krajewski, S., Armstrong, R., Wilson, G. M., Oltersdorf, T., Fritz, L. C., Red, J. C., and Ottilie, S. (1997). Bax- and Bak-induced cell death in the fission yeast Schizosaccharomyces pombe. Mol. Biol. Cell 8, 325-329.
Kohler, F. and Milstein, C. (1975). Continuous cultures of fused cells secreting antibody of predefined specificity. Nature 256, 495-497.
Ligr, M., Madeo, F., Froehlich, E., Hilt, W., Froehlich, K.-U. and Wolf, D. H. (1998). Mammalian Bax triggers apoptotic changes in yeast. FEBS Lett. 438, 61-65.
Lockhart, D. J. Dong, H. Byrne, M. C., Follettie, M., Gallo, M. V., Chee, M. S., Mitteman, M., Wang, C., Kobayashi, M., Horton, H and Brown, E. L. (1996). Expression monitoring by hybridisation to high density oligonucleotide arrays. Nature Biotechnology 14, 1675-1680.
Raff, M. C. (1992). Social controls on cell survival and cell death. Nature 356, 397-400.
RiboGene Inc., Patent application (1995) PCT WO 95/11969.
Sambrook J., Fritsch E. F. and Maniatis, T. (1989). Molecular Cloning: A Laboratory Manual, 2^{nd} Ed., CSH Laboratory Press, NY.
Sandbaken, M. G., Lupisella, J. A., DiDomenico, B., and Chakraburtty, K. (1990). Protein synthesis in yeast Structural and functional analysis of the gene encoding elongation factor III. J. Biol. Chem. 265, 15838-15844.
Sherman, F., Fink, G. R., and Hicks, J. B. (1979). Methods in yeast genetics, CSH Laboratory Press, NY.
Schiestl, R. H., and Gietz, D. R. (1989). High efficiency transformation of intact yeast cells using single stranded nucleic acids as a carrier. Curr. Genet. 16, 339-346.
Stoesser, G., Moseley, M. A., /Sleep, J., McGowran, M., Garcia-Pastor, M. and Sterk, P. (1998). Nucleic Acids Res. 26, 8-15.
Thomas, B. J. and Rothstein, R. J. (1989). Elevated recombination rates in transcriptionally active DNA. Cell 56, 619-630.
Walsh, T. J. (1992). Invasive Fungal Infections: Problems and Challenges for Developing New Antifungal Compounds, in: “Emerging Targets in Antibacterial and Antifungal Chemotherapy”, J. A. Sutcliffe and N. H. Georgopapadakou, eds, Chapman and Hall, NY, pp. 349-373.
Zhu, J. (1986). One step selection of a multicopy integrant based on yeast genomic transformation. In “Heterologous gene expression in Saccharomyces cerevisiae using a dominant selection and amplification system”. Ghent University, doctoral dissertation, p 45.

1. A genetically modified yeast or fungus comprising a nucleic acid encoding a protein having an amino acid sequence as represented in SEQ ID NO 2.

2. A genetically modified yeast or fungus comprising a nucleic acid molecule comprising a sequence as represented in SEQ ID NO 1.

3. The yeast or fungus of claim 1 wherein the yeast or fungus is selected from the group consisting of Saccharomyces cerevisiae, Schizosaccharomyces pombe, Candida albicans, or Aspergillus fumigatus.

4. The yeast or fungus of claim 3 wherein the nucleic acid is derived from Candida albicans or Saccharomyces cerevisiae.

5. An isolated nucleic acid molecule encoding a polypeptide having an amino acid sequence as represented in SEQ ID NO 2.

6. An isolated nucleic acid molecule comprising the sequence SEQ ID NO 1.

7. The nucleic acid molecule of claim 6 which is mRNA.

8. The nucleic acid molecule of claim 6 which is DNA.

9. The nucleic acid molecule of claim 6 which is cDNA.


SEQUENCE LISTING (2 sequences)

SEQ ID NO: 1 - DNA, 914 bp, Candida albicans
aaatacaaatccaagaaacctcgatgaggatgactctgatgataatgatgactctgatgagcgagagatttggtagattcaagccctctactatgttttatagttgacatatttgtatataaaaacttatacattattaaacatttgcgcgtcgattgacttttatttattattaacaaagaagtaataccaacctaattacaaatacttcgaagtgactatcataagtttccttatctagcgaaggcaacttttgaactccccagttgttaatatgtatcattatacacgacccaatcaaacgcggggaagtcaatgccgaaagaattctaggacctaaaagctgctcaatccttgggcctttccctaatgacatcccctctcaaactttagcttagcagttgtatttaatgtcctgtcacggatagtcaataatcgttgaaggttgattttcatatccttcgcaatttcgtaaagcaacaatagcaatacggactaaaatggtatgttggtgtgtgtgtgtgctgcttcacatttcaggctaaaaatgttatccgtggaatcttccttagccaagtatcatgggctttggaaattagtttcccaccgatgagcgcaacgacttagcgaagcttatgaaattggtgacagcagctctatgaatatgttccatgcgtttccattcaggttactaacaatgcataaattatgagtagtctttttcatcactatataaaacctttttcaaacgaaacgctgtttttgttggtactatctttgcaggtgcctttgttttccaaactgtatttgatactgctattacttcatggtacgagaatcacaacaaaggaaaattatggaaagatgtcaaggctcgaatagctgcaggcgatggagacgacgatgatgagtaa

SEQ ID NO: 2 - protein, 66 aa, Candida albicans
MSFSSLYKTFFKRNAVFVGTIFAGAFVFQTVFDTAITSWYENHNKGKLWKDVKARIAAGDGDDDDE